(19) United States
(12) Patent Application Publication
(10) Pub. No.: US 2012/0225460 A1
(43) Pub. Date: Sep. 6, 2012

(54) SYSTEM AND METHOD FOR PROCESSING BIOMASS

(75) Inventors: Mark T. Holtzapple, College Station, TX (US); Richard Davison, Bryan, TX (US); Cesar Granda, College Station, TX (US); Gary P. Noyes, Houston, TX (US); Edward Darlington, III, Belton, MO (US)

(73) Assignee: The Texas A&M University System, College Station, TX (US)

(21) Appl. No.: 13/473,565

(22) Filed: May 16, 2012

Related U.S. Application Data
(62) Division of application No. 12/708,298, filed on Feb. 18, 2010.
Application No. 12/708,298 is a continuation of application No. 11/298,983, filed on Dec. 9, 2005, now abandoned.
(60) Provisional application No. 60/635,235, filed on Dec. 10, 2004.

(51) Int. Cl. C12P 7/40

(52) U.S. Cl. 435/136

(57) ABSTRACT

A method for processing biomass that includes forming a first pile comprising biomass; inoculating said first pile comprising biomass; circulating a liquid in said first pile; fermenting the biomass in said first pile to produce a carboxylate salt; and extracting at least a portion of the liquid in the first pile and passing said portion of liquid to a second pile comprising biomass, wherein said second pile is operating at a higher carboxylate salt concentration than said first pile.

### CROSS-REFERENCE TO RELATED APPLICATIONS

This application is a divisional of U.S. patent application Ser. No. 12/708,298, filed Feb. 18, 2010, which is a continuation application of U.S. patent application Ser. No. 11/298,983, filed Dec. 9, 2005, which claims the benefit under 35 U.S.C. §119(e) of U.S. Provisional Patent Application No. 60/635,235, filed Dec. 10, 2004. The disclosures of said applications are hereby incorporated herein by reference in their entirety for all purposes.

### STATEMENT REGARDING FEDERALLY SPONSORED RESEARCH OR DEVELOPMENT

Not Applicable.

### BACKGROUND

1. Technical Field of the Invention

The present invention relates generally to biomass processing and, more specifically, a system and method for the storage, pretreatment, and fermentation of biomass.

2. Background of the Invention

Processing biomass, especially waste biomass, to recover useful substances has been the focus of numerous efforts. Such treatments have used a variety of treatment methods and chemicals depending upon the desired recovery substance. Treatment with lime has been attempted, but usually at temperatures above 60° C. for time frames of only a few weeks to a month. For example, previously issued patents to Holtzapple and Davison use high-temperature lime treatments to enhance enzymatic digestibility. One patent uses hot lime only and another uses hot lime plus high-pressure oxygen.

The most common methods for making pulp for paper or cardboard are Kraft and soda pulping. However, both of these methods use expensive chemicals and expensive treatment vessels. Additionally, previous methods and treatment systems often require movement of the biomass several times during the entire treatment process, including pretreatment and recovery.

### SUMMARY

According to one embodiment of the invention, a system for processing biomass comprises a chamber, a biomass input device, a fluid input device, and a retrieval device. The chamber is defined by at least a bottom, at least one wall, and a cover supported by the at least one wall. The biomass input device operable to deliver biomass into the chamber to form a biomass pile. The fluid input device is operable to deliver fluid into the chamber to the biomass pile. The retrieval device operable to receive fluid from the chamber.

Embodiments disclosed herein pertain to a method for processing biomass that may include forming a first pile comprising biomass; inoculating said first pile having biomass; circulating a liquid in said first pile; fermenting the biomass in said first pile to produce a carboxylate salt; and extracting at least a portion of the liquid in the first pile and passing said portion of liquid to a second pile having biomass, wherein said second pile may operate at a higher carboxylate salt concentration than said first pile.

The method may include introducing calcium carbonate into the first pile while said first pile comprising biomass is being formed. The method may also include circulating a liquid in said first pile is performed intermittently. In some aspects, the method may include pretreating the biomass prior to inoculating said first pile comprising biomass, wherein pretreating and fermenting said biomass take place in the same enclosure without transferring the biomass. In further aspects, pretreating the biomass may include introducing into the biomass lime or lime and air. In an embodiment, the air may be scrubbed of carbon dioxide prior to being introduced into the biomass.

The method of processing may include handling a multiplicity of piles comprising biomass in a round-robin manner. The method may also include regulating the temperature of the circulating liquid in said first pile. In an embodiment, regulating the temperature of the circulating liquid in said first pile may include passing said circulating liquid through a heat exchanger. In some aspects, the method includes controlling the temperature of the biomass in said first pile by regulating the temperature of the circulating liquid in said first pile.

Other embodiments of the present disclosure pertain to a method of processing biomass that may include the steps of transporting biomass to a chamber to form a biomass pile, the chamber defined by at least a bottom and adjustable cover; transferring a liquid to the chamber; lowering the adjustable cover from a raised position to a lowered position; fermenting the biomass pile in the chamber to produce a carboxylate salt while the adjustable cover is in the lowered position; and extracting at least a portion of the liquid from the biomass pile and passing the portion of liquid to a second pile comprising biomass, wherein said second pile is operating at a higher carboxylate salt concentration than the biomass pile.

The method may include fermenting the biomass in the presence of an inoculum. In an embodiment, the adjustable cover may be in the lowered position. The method may include supporting the adjustable cover with a pole that has perforations. In an embodiment, transporting the biomass to the chamber to form the biomass pile and transferring fluids to the chamber are carried out through the perforations. In other aspects, lowering the adjustable cover from a raised position to a lowered position may be carried out by a winch located on the pole, where the winch may be attached to the adjustable cover via a cable and releasing the cable to lower the adjustable cover.

Yet further embodiments of the disclosure pertain to a method for processing biomass that may include forming a first pile comprising biomass; inoculating the first pile; contacting a fluid with the first pile; fermenting the biomass in the first pile to produce a carboxylate salt; and collecting at least a portion of the fluid from the first pile, the fluid comprising at least some of the carboxylate salt, and passing the portion of fluid to a second pile comprising biomass.

The method may include introducing calcium carbonate into the first pile while said first pile comprising biomass is being formed. In some aspects, contacting the fluid with the first pile may be performed intermittently. In other aspects, the method may include pretreating the biomass prior to inoculating said first pile comprising biomass, wherein pretreating and fermenting said biomass take place in the same enclosure without transferring the biomass. In further aspects, pretreating the biomass may include introducing into the biomass lime or lime and air.

Certain embodiments of the invention may provide numerous technical advantages. For example, a technical advantage of one embodiment may include the capability to keep pretreators/fermentors at a low cost. Other technical advantages of other embodiments may include the capability to allow pretreatment and fermentation to occur in the same vessel. Yet other technical advantages of other embodiments may include the capability to remove spent solids.

Although specific advantages have been enumerated above, various embodiments may include all, some, or none of the enumerated advantages. Additionally, other technical advantages may become readily apparent to one of ordinary skill in the art after review of the following figures and description.

### BRIEF DESCRIPTION OF THE DRAWINGS

For a more complete understanding of example embodiments of the present invention and its advantages, reference is now made to the following description, taken in conjunction with the accompanying drawings, in which: FIG. 1 is a schematic of a system for processing biomass, according to an embodiment of the invention;

FIG. 2A-2D illustrate a system for processing biomass, according to another embodiment of the invention;

FIG. 3 shows a illustrate a system for processing biomass, according to another embodiment of the invention;

FIG. 4 shows a cutaway view of a system for processing biomass, according to another embodiment of the invention;

FIG. 5 shows a system for processing biomass, according to another embodiment of the invention;

FIG. 6 shows a system for processing biomass, according to another embodiment of the invention;

FIGS. 7A and 7B shows rigid covers, according to embodiments of the invention;

FIGS. 8A, 8B, and 8C show a system for processing biomass, according to another embodiment of the invention;

FIG. 9 shows a system for processing biomass, according to another embodiment of the invention;

FIG. 10 shows a system for processing biomass, according to another embodiment of the invention;

FIG. 11 shows a perspective view of a bottom of a chamber, according to an embodiment of the invention;

FIG. 12 shows a perspective view of a bottom of a chamber, according to an embodiment of the invention;

FIG. 13 shows multiple screw conveyors at a bottom of a chamber, according to an embodiment of the invention;

FIG. 14 shows a system for processing biomass, according to another embodiment of the invention;

FIGS. 15 and 16 show screw conveyors in V-shaped sections, operable to move material towards a conveyor, according to an embodiment of the invention;

FIG. 17 shows a system for processing biomass, according to an embodiment of the invention;

FIG. 18 shows a system for processing biomass, according to another embodiment of the invention;

FIG. 19 shows a system for processing biomass, according to another embodiment of the invention;

FIG. 20 shows a cone, according to an embodiment of the invention;

FIG. 21 shows a cone, according to an embodiment of the invention;

FIGS. 22 and 23 illustrates the use of a jet in conjunction with a cone, according to embodiments of the invention;

FIG. 24 shows a cone formed in a floor, according to an embodiment of the invention;

FIG. 25 shows a grate, according to embodiment of the invention;

FIG. 26 shows an isometric view of a cut-away cone, according to embodiment of the invention; and

FIGS. 27, 28, and 29 show patterns for cones, according to embodiments of the invention.

### DETAILED DESCRIPTION

It should be understood at the outset that although example embodiments of the present invention are illustrated below, the present invention may be implemented using any number of techniques, whether currently known or in existence. The present invention should in no way be limited to the example embodiments, drawings, and techniques illustrated below, including the embodiments and implementation illustrated and described herein. Additionally, the drawings are not necessarily drawn to scale.

As briefly identified in the Background, processing biomass, especially waste biomass, to recover useful substances has been the focus of numerous efforts. Accordingly, teachings of some embodiments recognize a system and method that converts biomass to carboxylic acids using a mixed culture of microorganisms. Further, teachings of some embodiments of the invention recognize an economical construction of biomass processing systems. Yet further, teachings of some embodiments of the invention recognize a system and methods for integrating pretreatment and fermentation into a single chamber or vessel. Additionally, teachings of some embodiments of the invention recognize a system and methods for removing spent solids from fermenting chambers.

Particular embodiment may be utilized to process biomass, for example, lignocellulosic biomass and other types of biomass, with lime or other alkali to yield useful recovery products. Other embodiments may be utilize other treatment methods. In addition, in some embodiments, the technology described herein may be utilized in conjunction with the technology described in U.S. patent application Ser. No. 10/698,199, filed Oct. 31, 2003 which is herein incorporated by reference.

FIG. 1 is a schematic of a system 100 for processing biomass 105, according to an embodiment of the invention. The system 100 of FIG. 1 presents example components that may be utilized in such a processing of biomass. For purposes of brevity, the structural details of various components of system 100 are not shown or described. For example, the system may include a cover, which is not shown in FIG. 1. Additionally, although specific components are shown with reference to the system 100 of FIG. 1, other systems may utilize more, fewer, or different component parts.

In the embodiment of FIG. 1, the system 100 includes a water-impermeable bottom liner 102, a gravel layer 104, a drainage device 106, a perforated pipe 107, a biomass input device 108, a lime input device 110, a calcium carbonate input device 112, a distribution device 114, a perforated pipe 115, a pump 116, a water supply 118, an inoculum supply 120, an air distribution device 122, a perforated conduit 123, an air blower 124, a lime water slurry container 126, and a heat exchanger 128. In particular embodiments, the system 100 may be utilized as a multi-use facility, which accepts and stores untreated biomass, pretreats the biomass, and ferments the biomass. Such a multi-use facility in particular embodiments may result in a reduction of biomass handling.

The liner 102 in particular embodiments may be formed of a water-impermeable material. In operation, the liner 102 supports the gravel layer 104 and prevents water or other materials from entering the ground. The liner 102 may be placed upon any suitable support. In the embodiment of FIG. 1, the liner 102 is shown in a pit or bermed wall in the ground. The liner 102 may have any suitable shape and the depth. In particular embodiments, the liner 102 may be designed to handle a desired amount of gravel for the gravel layer 104. An example depth for gravel layer 104 is approximately three feet; however, other suitable depths may also be utilized for gravel layer 104. The gravel layer 104 may be comprised of any suitable loose or unconsolidated deposit of rounded pebbles, cobbles, boulders, or other suitable stone-like material that allow water to flow relatively freely therethrough.

Shown disposed on top of the gravel layer 104 in this embodiment is a pile of biomass 105 that may be delivered over gravel layer 104 via biomass input device 108. Biomass input device 108 represents any suitable device for creating biomass pile 105, such as a suitable conveyer system, front-end loader, or other suitable delivery system or device. As described above, the biomass in one embodiment, is lignocellulosic biomass, such as bagasse, corn stover, or other suitable biomass.

The lime input device 110 and the calcium carbonate input device 112 are any suitable devices operable to deliver lime and calcium carbonate, respectively, to the pile of biomass 105. In particular embodiments, the lime and/or calcium carbonate may be delivered while the pile of biomass 105 is formed so that the materials are evently distributed throughout. In other embodiments, lime and/or calcium carbonate may utilized to pretreat the biomass. Although the amount of lime added to the pile of biomass 105 may vary depending on the type of biomass 105, in one embodiment, an amount of lime delivered to the pile of biomass 105 is between approximately 10% and 30% of the biomass by weight.

Water from water supply 118 may be circulated through biomass pile 105 by pump 116 by delivering the water through distribution device 114, which may be any suitable device operable to distribute the water to the biomass pile 105. In particular embodiments, the distribution device 114 may include a perforated pipes 115 while in other embodiments the distribution device 114 may be spray head(s) or other suitable devices. After the water has traveled through the biomass pile 105 and gravel layer 104, the water is recovered through a drainage device 106, which may include a perforated pipe 107. In such an operation, circulation of the water may either be continuous with a relatively low flow rate or may be intermittent with a relatively high flow rate.

With a continuous circulation and low flow rate, channeling may occur which is undesirable because some portions of biomass pile 105 may not be wetted. Uneven wetting of biomass pile 105 may cause any one or more of the following problems: incomplete pretreatment of the pile of biomass 105, poor temperature control, and spontaneous combustion of dried portions of the pile of biomass 105. An intermittent circulation and high flow rate periodically floods the pile of biomass 105, thus ensuring all or most portions are wetted, thereby overcoming the potential problems of continuous circulation with low flow rate.

The temperature of the water circulated through the pile of biomass 105 may be regulated with the heat exchanger 128. The heat exchanger 128 may be any suitable device used to control the temperature of the water circulated through biomass pile 105. For example, heat exchanger 128 may be a shell-and-tube type heat exchanger designed to offload thermal energy.

While biomass pile 105 is being pretreated, air may be blown upward through biomass pile 105 to enhance lignin removal by alkaline oxidation. This may be facilitated by air blower 124 forcing air through air distribution device 122, which may include in particular embodiments a perforated conduit 123 disposed inside the gravel layer 104. Because air contains carbon dioxide, it may react with lime to form calcium carbonate, an unproductive reaction. To prevent this from occurring in biomass pile 105, the air may be scrubbed of carbon dioxide by passing it through lime water slurry in container 126, which may be a suitable packed column or tank. Oxygen enriched air flow may also be used.

The pile of biomass 105 may be subjected to a fermentation process while disposed over gravel layer 104. To facilitate the fermentation after pretreatment is complete, water may be circulated through biomass pile 105 that contains an inoculum of acid-forming microorganisms obtained from inoculum supply 120. The acid-forming microorganism start to degrade pile of biomass 105, forming carboxylic acids that react with calcium carbonate to form calcium carboxylate salts. Water may then be circulated through the pile of biomass 105 to remove the carboxylate salts.

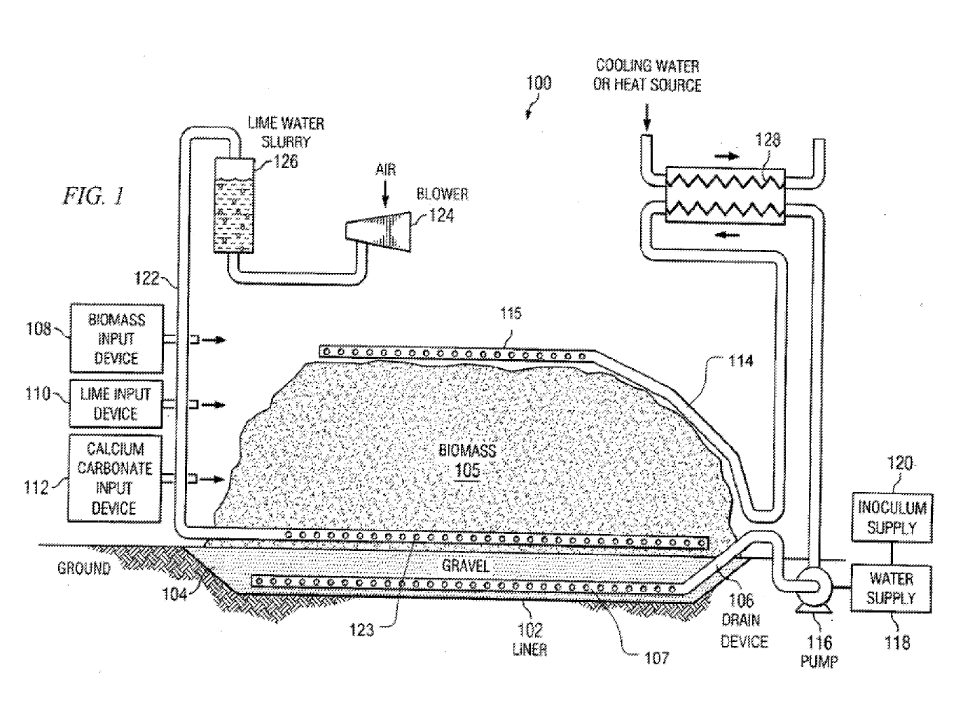

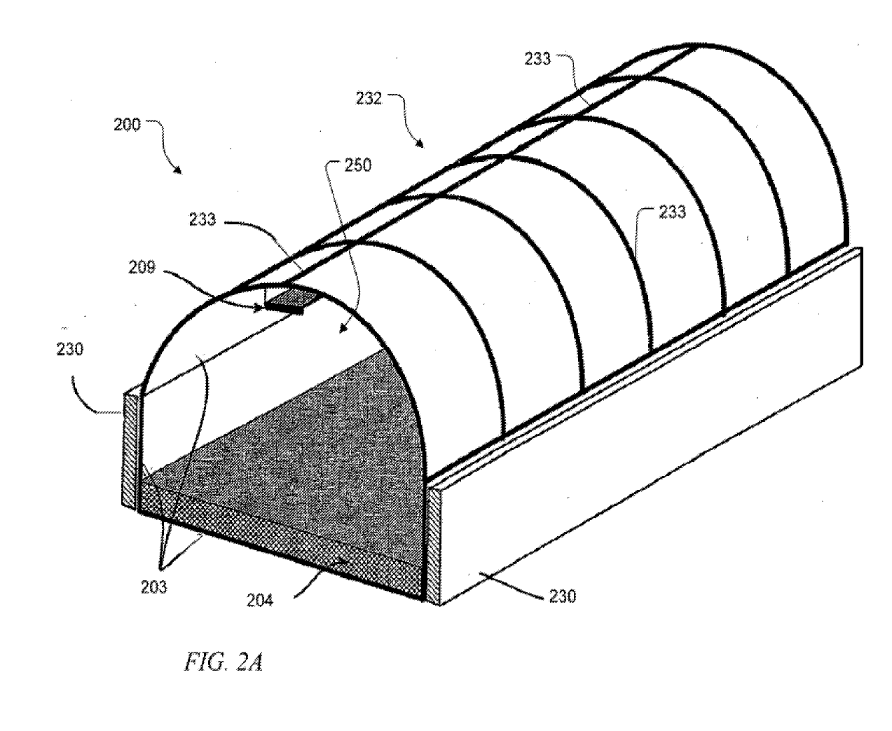

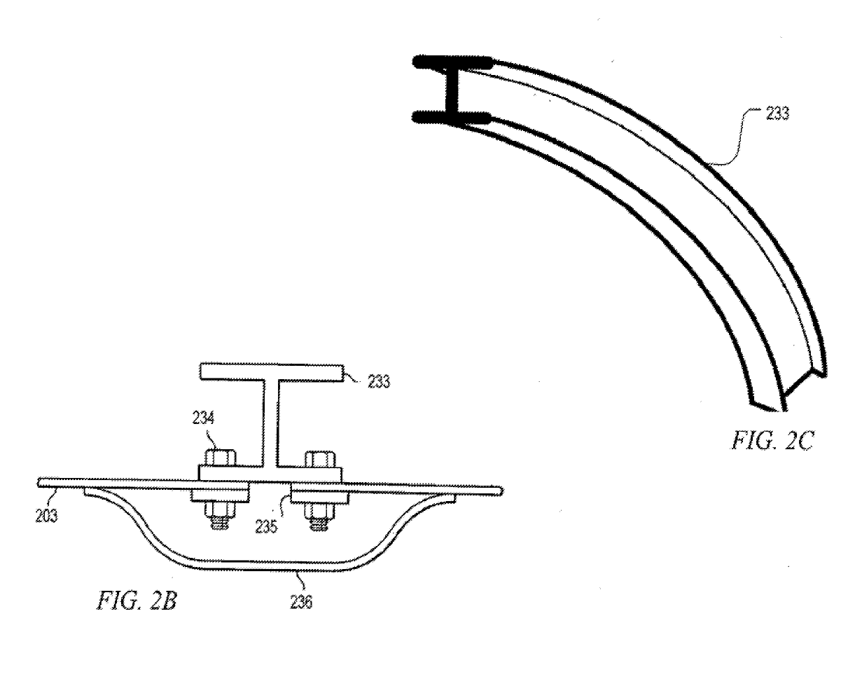

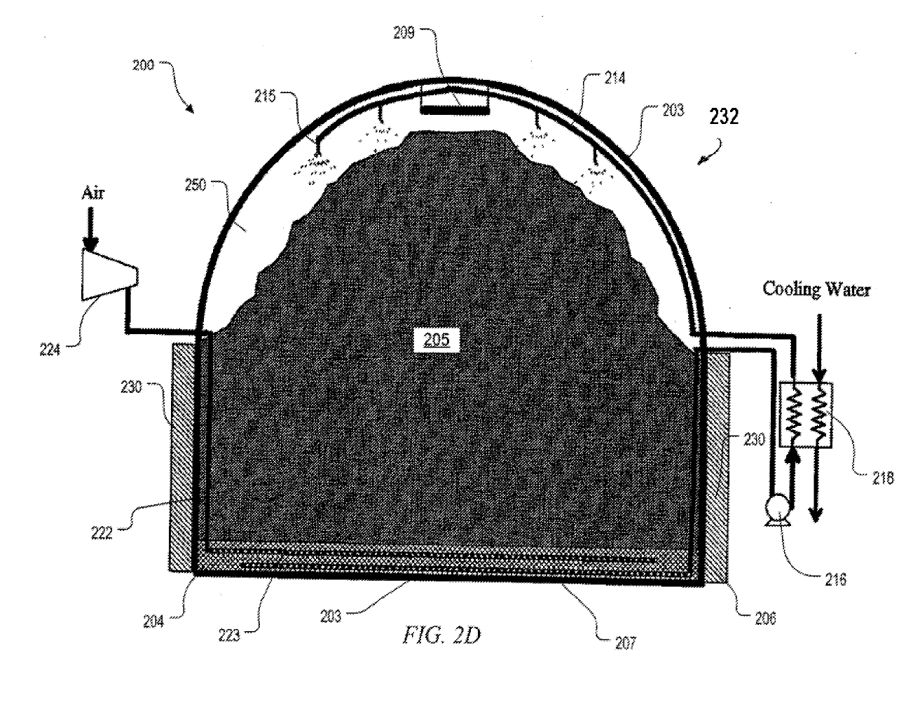

The storage, pretreatment, and fermentation of biomass may also be accomplished using other suitable storage facilities or systems. Various embodiments of these systems are described below in conjunction with FIGS. 2A-29. The components described with reference to FIG. 1 may be utilized in conjunction with any of the systems described with reference to FIGS. 2A-29.

FIG. 2A-2D illustrate a system 200 for processing biomass 205, according to another embodiment of the invention. FIG. 2A shows an isometric view of a portion of the system 200. The system 200 is similar to the system 100 of FIG. 1 except that system 200 includes a geomembrane 203, a cover 232, support ribs 233, walls 230, and a conveyor 209. As described in further details below, the various components of the system 200 may form a chamber 250 that can be used for storing untreated biomass, pretreating the biomass, and fermenting the biomass.

The geomembrane 203 may be formed from any suitable material and may perform a similar function to the liner 102 of FIG. 1. In particular embodiments, the geomembrane 203 may line a substantial portion of the chamber 250. The geomembrane 203 in this embodiment is disposed beneath a gravel layer 204 on a bottom portion of the system 200. The geomembrane 203 lines the interior of the walls 230 and extends over chamber 250 with support from the support ribs 204 to form the cover 232.

The walls 208 in this embodiment may be made of concrete. In other embodiments, the walls 208 may be made of other suitable materials. In particular embodiments, the walls 208 may extend above a ground level. In other embodiments, the walls may extend into the ground.

The support ribs 233 may be any suitable structure that can provide support for geomembrane 203 to help form the cover 232. For example, with reference to FIG. 2C, the support ribs 233 are shown as an I-beam. However, the support ribs 233 may also be other suitable structural members such as a lightweight truss.

The geomembrane 203 may be coupled to the support ribs 233 in any suitable manner. FIG. 2B illustrates one embodiment of coupling the support ribs 233 (shown as an I-Beam in this embodiment) to the geomembrane 203. With reference to FIG. 2B, one or more bolts 234 are utilized to couple geomembrane 203 to the support ribs 233. Other suitable fasteners other than bolts may also be utilized to couple geomembrane 203 to the support ribs 233. A pair of stiffener plates 235 may provide stiffness to geomembrane 203, which is disposed between the stiffener plates 235 and the support ribs 233 and coupled therebetween by bolts 234. To prevent the corrosion of the bolts 234 and the stiffener plates 235, a boot 236 formed from the similar or different material than the geomembrane 203 may be utilized to cover the bolts 234 and stiffener plates 235.

The conveyor 209 may operate to dispose the biomass 205 (seen in FIG. 2D) on top of the gravel layer 204 in the chamber 250. The conveyor 209 may be supported by the support ribs 233, running along the length of the system 200.

FIG. 2D shows a cross-sectional view of the system 200 of FIG. 2A, illustrating additional details of system 200 according to an embodiment of the invention. Biomass 205 is shown disposed on top of the gravel layer 204 on a bottom portion of the system 200. A perforated pipe 223 of an air distribution device 222 is embedded into the gravel layer 204 allowing compressed air from the air blower 224 to be blown up through the pile of biomass 205. Also, a perforated pipe 207 of the drainage device 206 is embedded into the gravel layer 204 to allow liquid to be pumped via pump 216 from the gravel layer 204 into sprayers 215 of distribution system 214 to wet the top of the pile of the biomass 205. In a manner similar to that described above with reference to FIG. 1, during the pretreatment phase, air may be blown up through the pile of the biomass 205 while water is simultaneously trickled through the pile of the biomass 205. The action of air plus lime (which may be premixed into the biomass pile) removes lignin from the biomass, rendering it more digestible. When the lime is exhausted, the pH drops near neutrality. At this point, an inoculum of mixed acid-forming microorganisms may be added (e.g., using an innoculum supply 120 such as that shown in FIG. 1), which digests the biomass 205 and converts it to mixed carboxylic acids. The acids react with calcium carbonate (which may be premixed into the biomass pile 205) to form carboxylate salts. The pump 216 circulates water through the pile of the biomass 205 to help extract the carboxylate salts as they are formed. During the above operations, the circulating liquid may go through a heat exchanger 228 to regulate temperature. Additionally, a portion of the circulating liquid may be passed to an adjacent chamber 250, which operates as a fermentor, that is operating at a higher carboxylate salt concentration. The circulating fluid may additionally be harvested and processed to recover the soluble product.

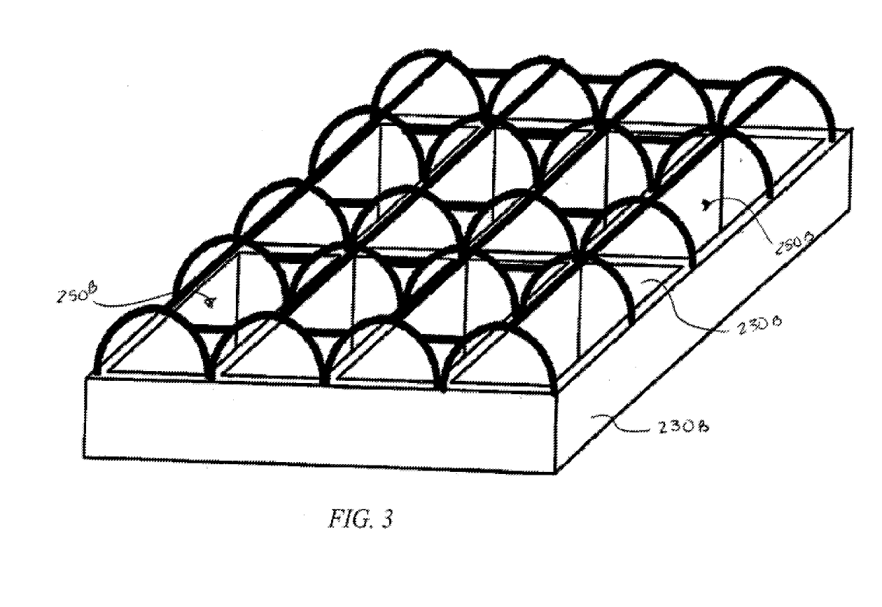

FIG. 3 shows a illustrate a system 200B for processing biomass, according to another embodiment of the invention. The system 200B comprises eight-cells or chambers 250B. For purposes of brevity, the system 200B is shown in partial view with only particular components shown (e.g., walls 230B) and some components ghosted. However, each of the cells or chambers 250B may operate in a similar manner to the chambers 250 of system 200 described above. For example, each of the chambers 250B may operate to store untreated biomass, pretreat the biomass, and ferment the biomass. Additionally, each chamber 250B of system 200B of FIG. 3 may include similar or different components than system 200. Further, each of the chambers 250B may operate as independent systems or systems that communicate with one another. The cells or chambers 250B may additionally be operated in a “round robin” manner. That is, one of the cells or chambers 250B would be in the process of filling with fresh biomass while another cell or chamber 250B would be in the process of removing spent solids. The other six cells or chambers 250B may be fermenting, each in a successive stage of digestion.

FIG. 4 shows a cutaway view of a system 200C for processing biomass, according to another embodiment of the invention. The system 200C of FIG. 4 shows how earth-moving equipment can enter a chamber 250C to remove spent solids in embodiments of the invention. In a manner similar to the system 200B of FIG. 3, the system 200C of FIG. 4 is in partial view with only particular components shown and some components ghosted. The system 200C in particular embodiments may include an elevated slop 297C, a wall 230C, a door 298C, and a slope passage 299C.

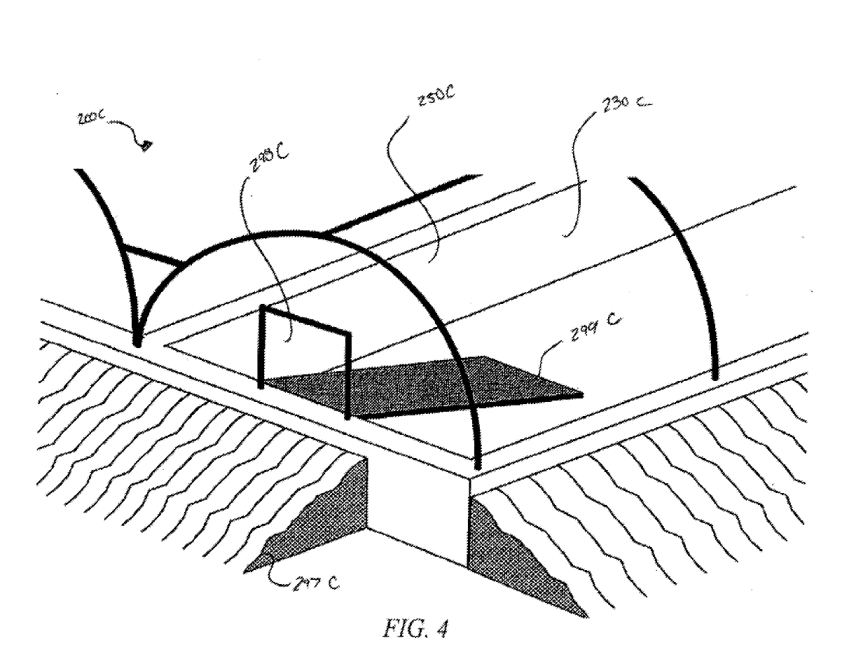

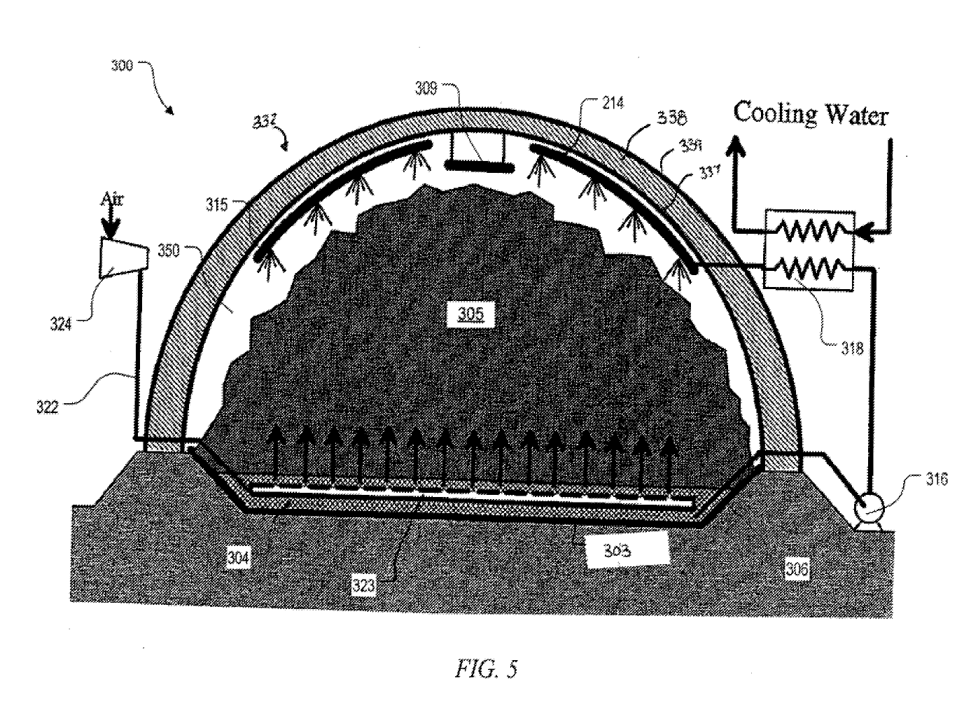

FIG. 5 shows a system 300 for processing biomass 305, according to another embodiment of the invention. The system 300 is similar to system 200 of FIGS. 2A-2D, including a geomembrane 303, a gravel layer 304, a pile of biomass 305, a chamber 350, an air blower 324, an air distribution device 322 with a perforated pipe 323, a conveyor 309, a drainage device 306, a pump 316, a distribution system 314 with sprayers 315, a cover 332, a chamber 350, and a heat exchanger 328. The system 300 is different in that the system 300 does not include walls and the cover 332 is a rigid cover. The rigid cover 332 in this embodiment is shown with an inner core 338 between an interior layer 338 and an exterior layer 339. The inner core 338 may be made from a variety of materials, including, but not limited to “papercrete,” a mixture of cement, sand, and paper pulp. The portions of each component of the papercrete can vary, but in particular embodiments includes the following mixture: cement 20%; sand 20%; and paper 60%. In other embodiments, the inner core 338 may be made of a papier mâché, a mixture of paper pulp and glue, or other suitable materials. The advantage of papercrete and papier mâché is that they can contain waste paper, which renders inner core 338 inexpensive. Also, the paper may act as an insulator, which helps regulate the temperature of the biomass pile. Because the paper component can be damaged when wetted, the inner layer 338 and the exterior layer 339 of the rigid cover 332 in some embodiments may be coated with a water-proofing material, such as tar.

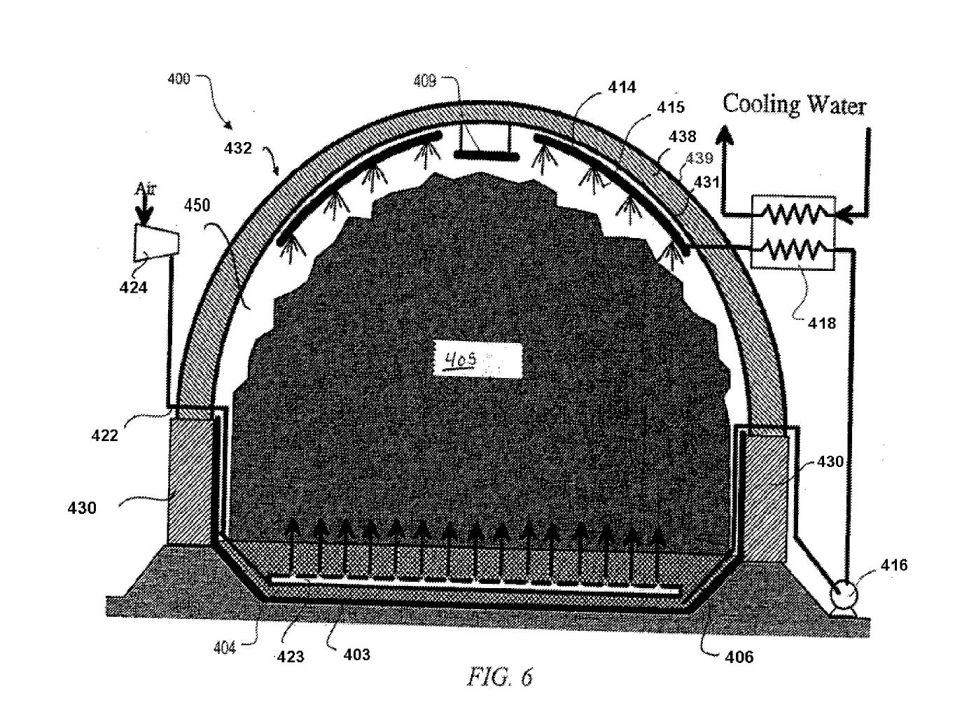

FIG. 6 shows a system 400 for processing biomass 405, according to another embodiment of the invention. The system 400 is similar to system 300 of FIG. 5, including a geomembrane 403, a gravel layer 404, a pile of biomass 405, a chamber 450, an air blower 424, an air distribution device 422 with a perforated pipe 423, a conveyor 409, a drainage device 406, a pump 416, a distribution system 414 with sprayers 415, a heat exchanger 428, a chamber 450, and a rigid cover 432 having an inner core 438 sandwiched between an interior layer 437 and an exterior layer 439. The system 400 of FIG. 6 additionally includes a concrete wall 430 surrounding a base of the system 400.

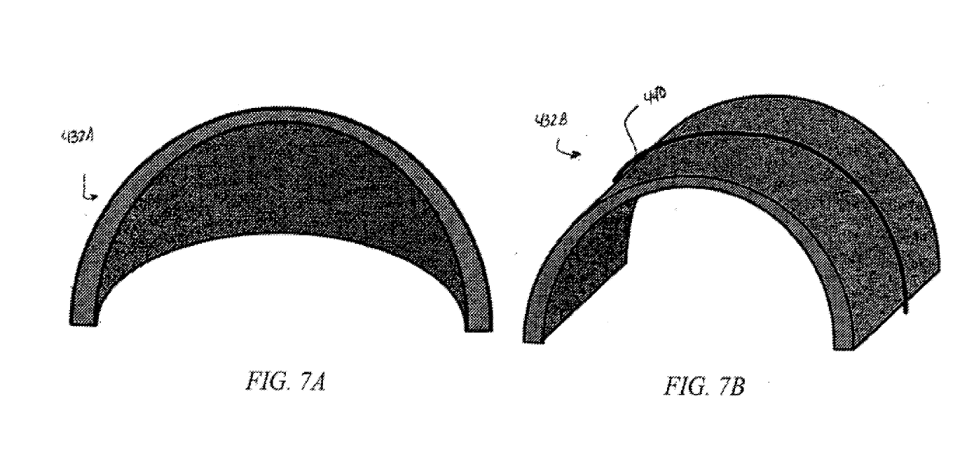

FIGS. 7A and 7B shows rigid covers 432A, 432B, according to embodiments of the invention. FIG. 7A show a cut-away view of a domed rigid cover 432A that may be used with a circular chamber 450 and FIG. 7B show a cut-away view of an arched rigid cover 432B that may be used with a rectangular chamber. Because the construction material is very light and could be blown over by the wind, the rigid covers 432A, 432B may be secured to the ground using a cable 440. The rigid covers 432A, 432B shown with reference to FIGS. 7A and 7B may be utilized with various embodiments of the invention.

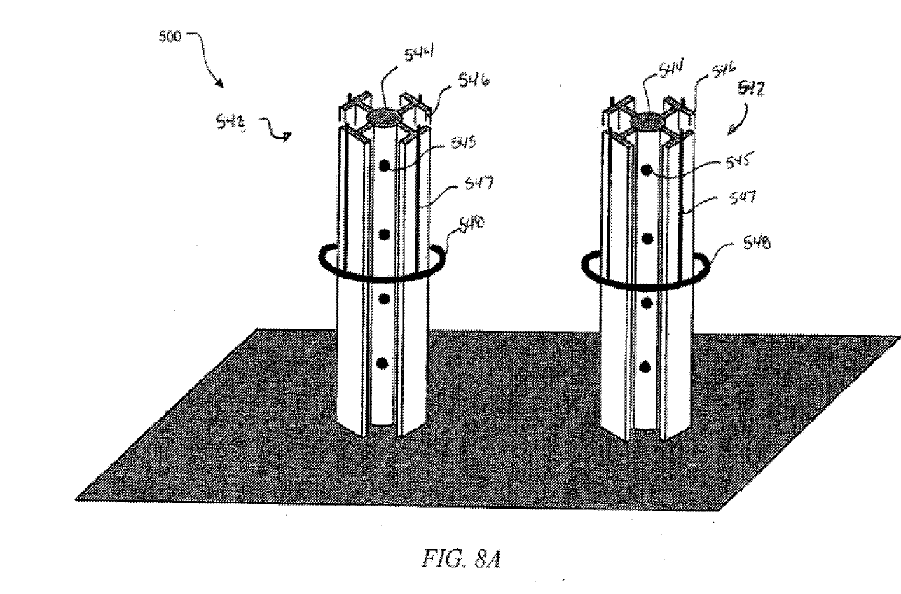

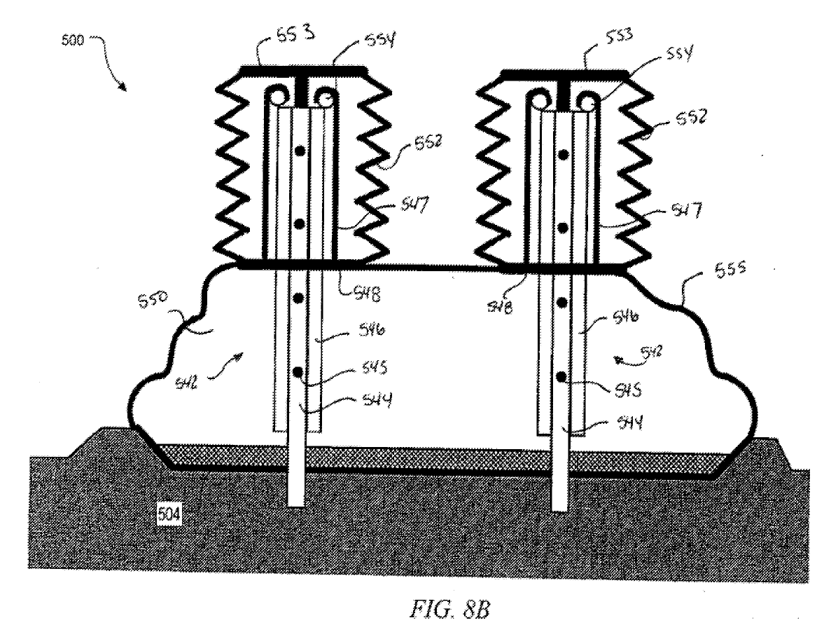

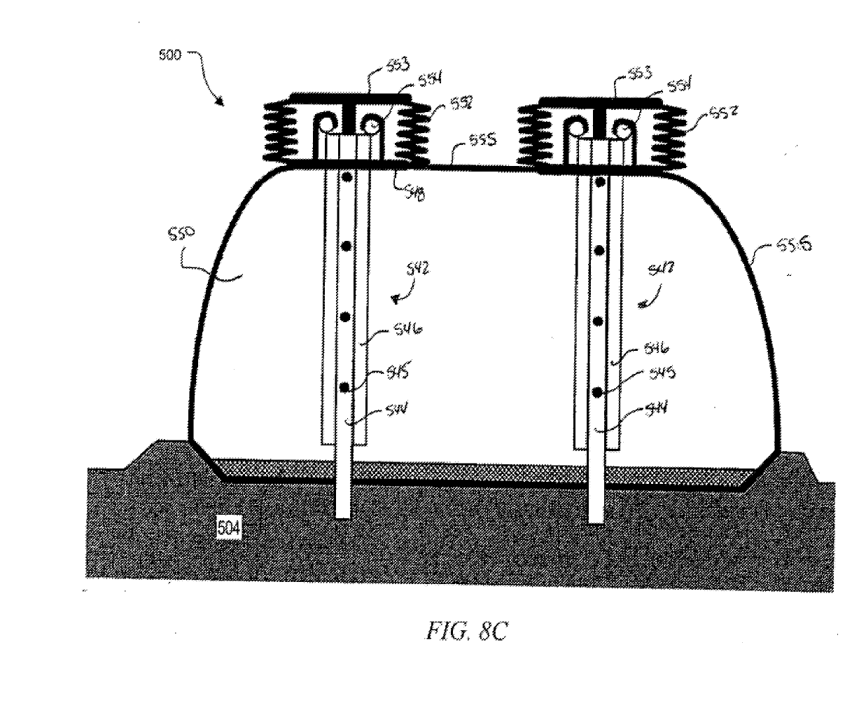

FIGS. 8A, 8B, and 8C show a system 500 for processing biomass, according to another embodiment of the invention. FIG. 8A show tent poles 542 for the system 500. The tent poles 542 are shown protruding from a gravel layer 504. In this embodiment, the tent poles 542 include pipes 544 at the center with I-beams 546 welded to the exterior of the pipes 544. A hoop 548 surrounds the pole 544, which can be raised or lowered using a winch system (seen in FIG. 8B), which can pull on cables 547. The pipes 544 may include perforations 545 at various levels. The perforations 545 allow a biomass pile to be constructed by pumping an aqueous slurry of the biomass through the pipes 544. In such an operation, the water carries the biomass to the pile and then drains away from the gravel base. The perforations 545 may also be used to circulate water through the pile during pretreatment or fermentation. Also, the perforations 545 may be used to deliver inoculum to the pile when the pretreatment is completed.

FIG. 8B shows the tent poles 542 supporting a flexible cover 555, which is in the low position. The flexible cover 555 may be made of a variety of materials. A bellows 552, extending from a support 553 provides flexibility as the flexible cover 555 is raised and lowered. In particular embodiments, the bellows 552 may facilitate a substantial enclosure of the chamber 550. During fermentation, the flexible cover 555 can be lowered onto the biomass pile. A slight vacuum can be applied to the chamber 550 by sucking away fermentation gases. This ensures that the flexible cover 555 is sucked tightly against the pile, which prevents it from being damaged by the wind.

FIG. 8C shows the flexible cover 555 in the raised position. The flexible cover 555 can be raised while the pile is being built, or when the solid residues are being removed. In such a raising of the flexible cover 555, the winch 554 can pull in the cables 547, which are attached to the rings 548, which are attached to the flexible cover 555.

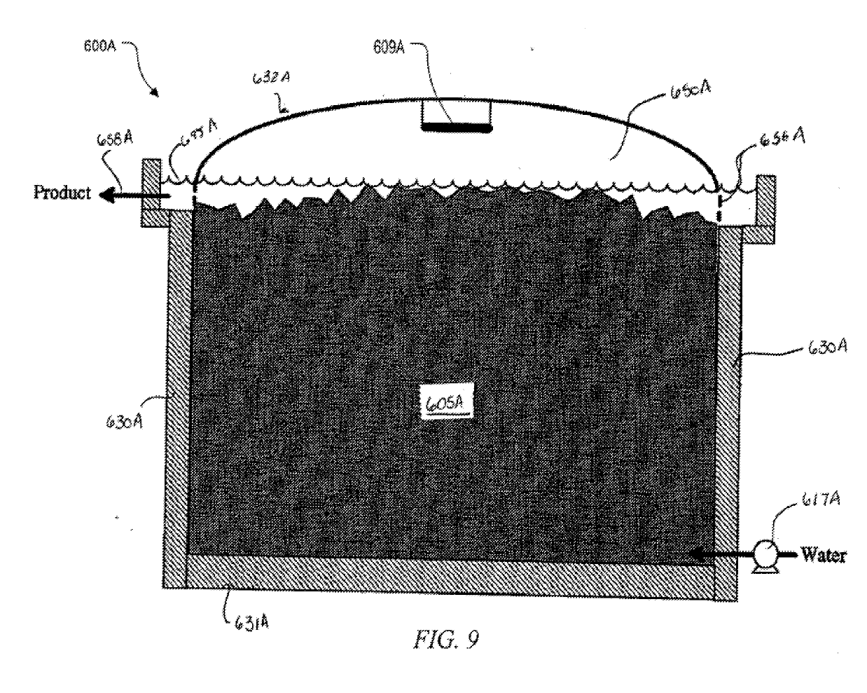

FIG. 9 shows a system 600A for processing biomass 605A, according to another embodiment of the invention. The system 600A may be similar to system 400 of FIG. 6. However, in the system 600A of FIG. 9, fresh water is added via a pump 617A at the bottom through a distributor system so that it percolates up through the biomass 605A, which is submerged under a water line 695A. Fresh biomass 605A may constantly be added at the top of a chamber 650A, for example, through a conveyor 609A or other suitable mechanism and spent biomass may be removed from the bottom, using embodiments described herein or any suitable mechanism. Product with carboxylate salts may be removed from the top at the area indicated by arrow 658A, using any suitable device. In particular embodiments, a screen 656A may be employed to keep solids from entering the liquid product stream. A cover 632A for the system 600A may be any suitable cover, including rigid covers, flexible covers, and others. Walls 630A and a floor 631A may be made from any suitable material, including but not limited to concrete.

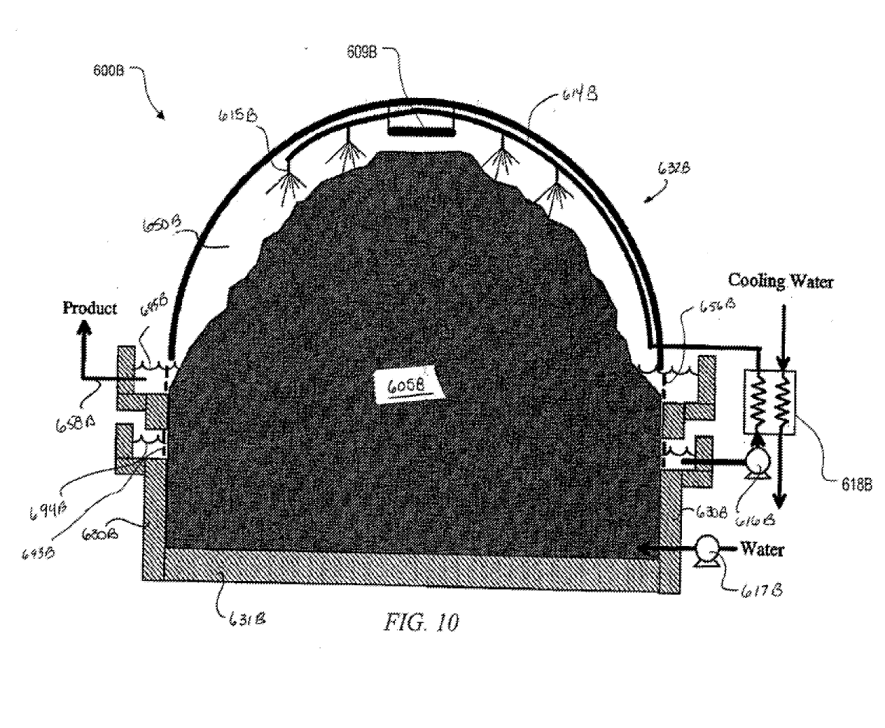

FIG. 10 shows a system 600B for processing biomass 605B, according to another embodiment of the invention. The system 600B is similar to the system 600A of FIG. 9 except that the system 600B has a significant portion of the biomass 605B located above a first water line 695B. Liquid may be removed from a second water line 694B just below a first water line 695B, heat exchanged with a heat exchanger 628B to regulate temperature, and pumped via a pump 616B onto the sprayers 615B located in a distribution system 614B of an arched or domed cover 632B. The spray percolates through the biomass 605B above the first water line 695B. Product with carboxylate salts may be removed from the top at the area indicated by arrow 658B, using any suitable device. Screens 656B, 693B may allow removal of liquid while preventing passage of solids. The cover 632B for the system 600B may be any suitable cover, including rigid covers, flexible covers, and others. Walls 630B and a floor 631B may be made from any suitable material, including but not limited to concrete.

FIG. 11 shows a perspective view of a bottom of a chamber 650, according to an embodiment of the invention. The bottom of the chamber 650, for example, may be the bottom of chambers 650A, 650B of FIGS. 9 and 10. The bottom of the chamber 650, which as referenced above may be used as a fermentor, is shown as a series of V-shaped sections 660 with a screw conveyor 662 located at the tip of the V-shaped sections. In one embodiment, the slope of the “V” is greater than the angle of repose, allowing solids to flow toward the top of the V.

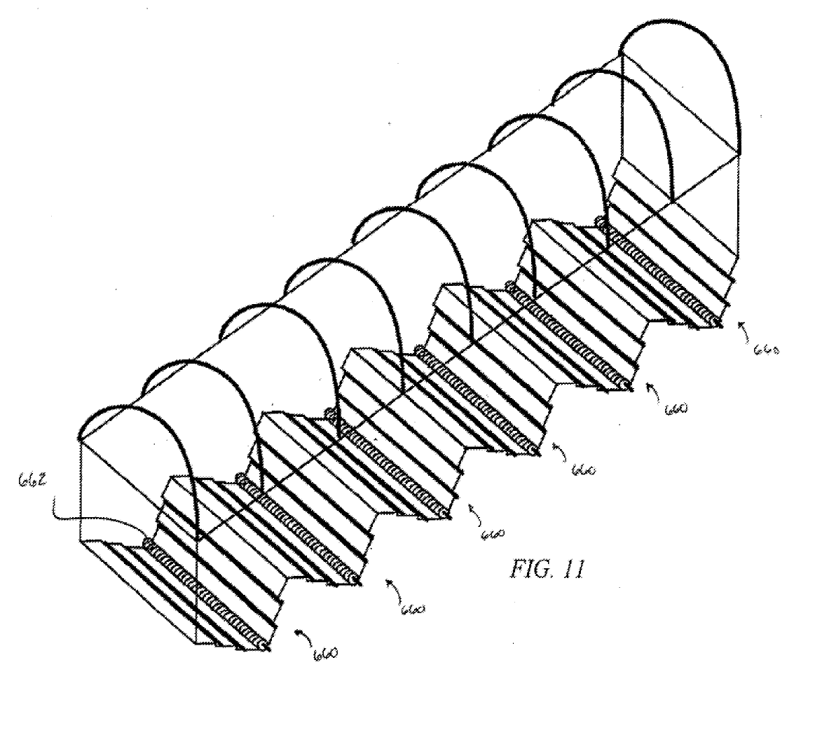

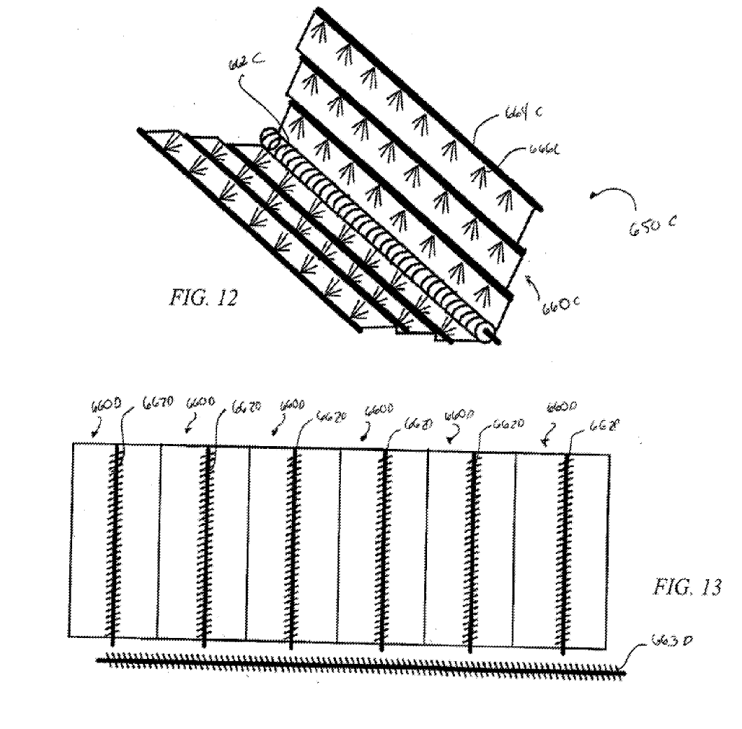

FIG. 12 shows a perspective view of a bottom of a chamber 650C, according to an embodiment of the invention. To help lubricate the surface and force solids downward towards the tip of a V-shaped section 660C, the section 660C may have water distributors 664C that employ nozzles 666C that blast liquid downward, forcing the solids downward.

FIG. 13 shows multiple screw conveyors 662D at a bottom of a chamber 650D, according to an embodiment of the invention. The screw conveyors 662 of the embodiment of FIG. 13 convey spent solids to a side, where another conveyor 663D removes them for ultimate disposal.

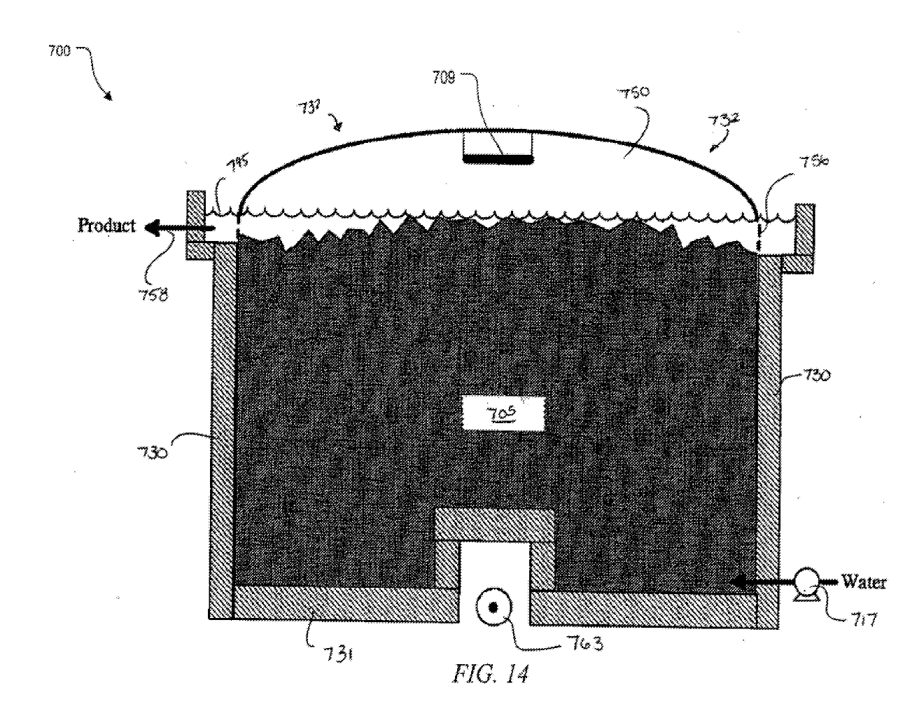

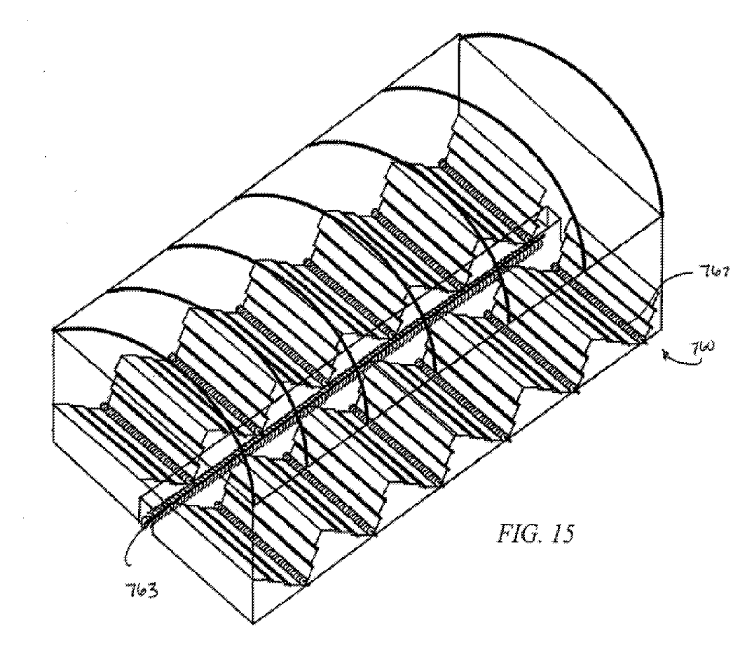

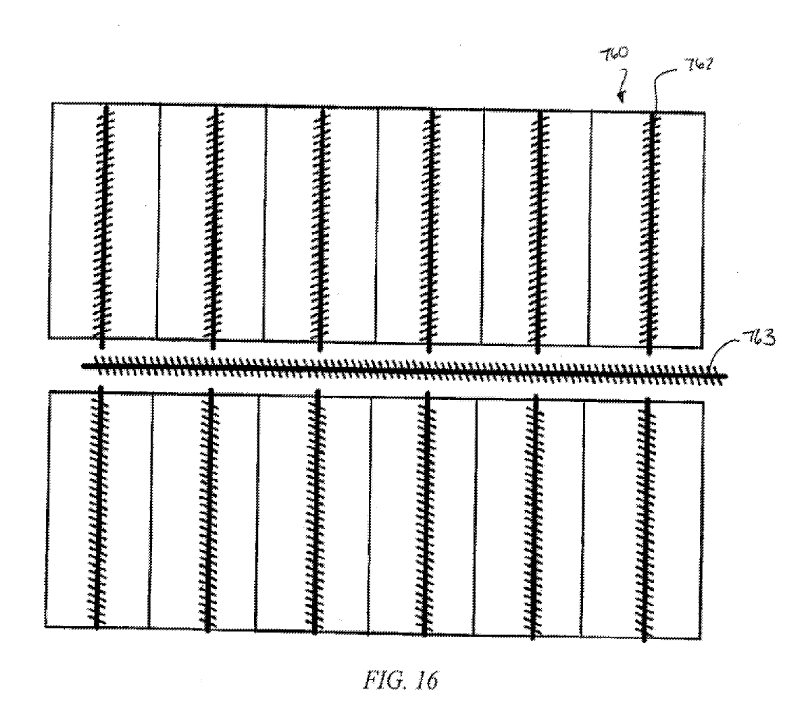

FIG. 14 shows a system 700 for processing biomass, according to another embodiment of the invention. The system 700 of FIG. 14 is similar to the system 600A of FIG. 9, including walls 730B, a floor 731B, a water line 795B, a pump 717, a screen 756, biomass 750, product removal indicated by arrow 758, a cover 732, and a water line 795. However, system 700 includes a conveyor 763 that removes solids for ultimate disposal is located at the center of the chamber 750 rather than the side. The conveyor 763 is serviced by screw conveyors 760 on both sides, which is readily seen in FIGS. 15 and 16.

FIGS. 15 and 16 show screw conveyors in V-shaped sections 760, operable to move material towards a conveyor 763, according to an embodiment of the invention.

FIG. 17 shows a system 800A for processing biomass 805A, according to an embodiment of the invention. The system 800A of FIG. 17 is similar to the system 600B of FIG. 10, including components such as walls 830A, a floor 831A, a first water line 895A, a second water line 894A, a heat exchanger 828A, a pump 816A, a pump 817A, a first screen 856A, a second screen 893A, sprayers 815A located in a distribution system 814A, product removal indicated by arrow 858, and a cover 832A. However, the system 800A has a single large cone 892A at the bottom of the chamber 850A that collects all spent solids. Further, the pump 817A pumps into a distribution device 888A. The biomass 805A may be introduced into the system using any of a variety of biomass input devices 808A. And, a conveyor 867A removes spent solids from the tip of the cone 892A and conveys them out the top of the cover 832A. To provide a back-up barrier that would prevent leakage of contents of the chamber 850A into the groundwater should a leak develop, the floor 831A and/or wall 830A that help define the chamber 850A may be located in a hole 890A that is filled with a gravel layer 804A and lined with a geomembrane 803A. The space between the floor 831A and/or wall 830A that help define the chamber 850A and the geomembrane 803 may be filled with water 891A, which provides hydraulic head and helps balance the pressure of the liquid inside the chamber 850A. Such a configuration may allows thinner concrete structures, for example, in the floor 831A and/or walls 830A. Additionally, such a configuration may eliminate the need for prestressing the concrete with steel cables that keep the concrete in compression, where it is strongest.

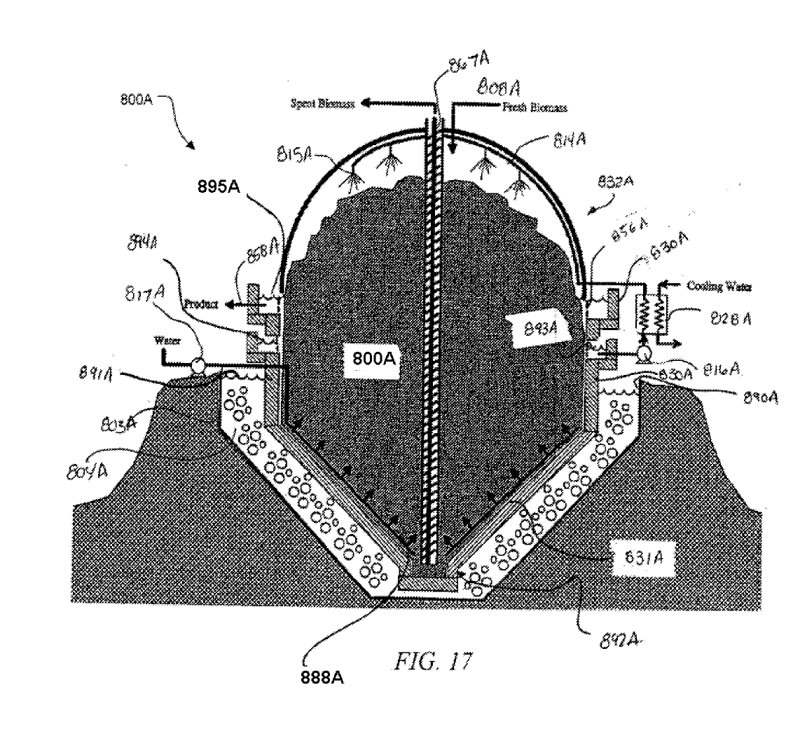

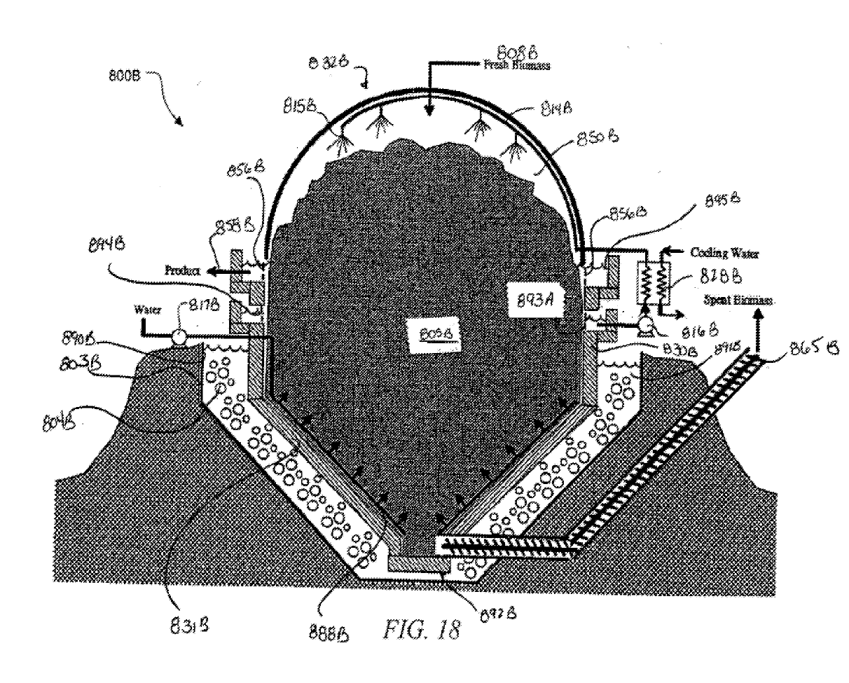

FIG. 18 shows a system 800B for processing biomass 805B, according to another embodiment of the invention. The system 800B of FIG. 18 is similar to the system 800A of FIG. 17, including components such as walls 830B, a floor 831B, a first water line 895B, a second water line 894B, a pump 816B, a heat exchanger 828B, a pump 817B that pumps water into a distribution device 888B, a first screen 856B, a second screen 893B, sprayers 815B located in a distribution system 814B, biomass 805B, product removal indicated by arrow 858B, a cover 832B, a cone 892B at the bottom of the chamber 850B, a biomass input device 808B, a hole 890B, a gravel layer 804B, a geomembrane 803B, and water 891B. However, a conveyor 865B removes spent solids from the tip of the cone 892B and removes them from a side of the system 800B.

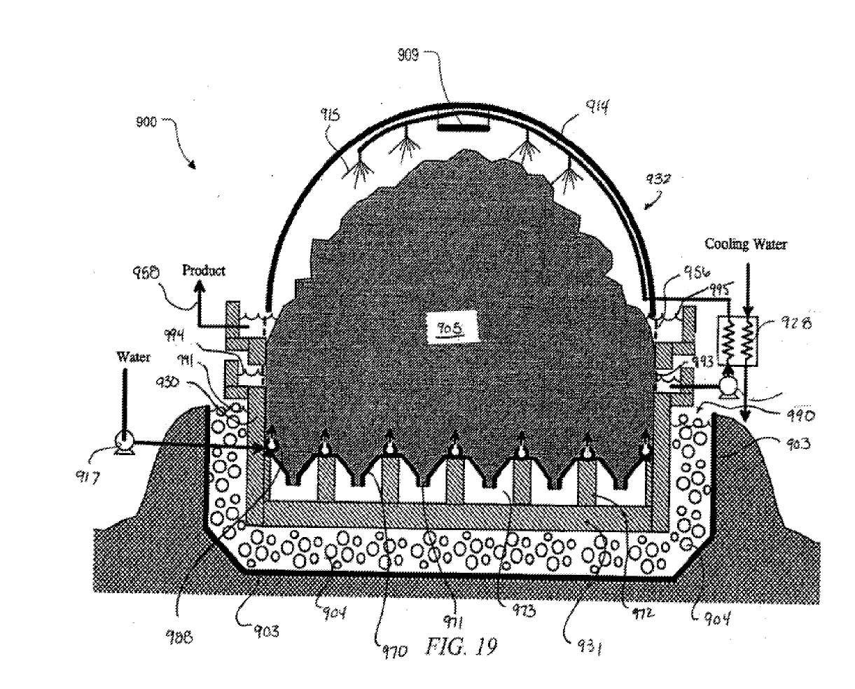

FIG. 19 shows a system 900 for processing biomass 905, according to another embodiment of the invention. The system 900 of FIG. 19 is similar to the system 800A of FIG. 17, including components such as walls 930, a floor 931, a first water line 995, a second water line 994, a pump 916, a heat exchanger 928, a pump 917 that pumps into a distribution device 988, a first screen 956, a second screen 993, sprayers 915 located in a distribution system 914, product removal indicated by arrow 958, a cover 932, a hole 990, a hole 990, a gravel layer 904, a geomembrane 903, and water 991. Biomass 905 may be delivered to the chamber 950 using a conveyor 909 or other suitable device. The system 900 includes multiple cones 970 for removing spent solids. Each of the cones 970 have a passage 971 at the tip and are supported by concrete ribs 972 extending from the floor 931. In particular embodiments, a space 973 created by the concrete ribs 972 may be large enough to allow a person to perform maintenance under the cones 970. In particular embodiments, the space 973 between the ribs 972 may have water circulating so that as solids flow through the cone 972, they are dispersed into a dilute slurry, which is easily pumped. When the dilute slurry is brought to the surface, the dilute slurry may be sent to a settling tank (not shown), where the solids settle out and the liquid is recovered for recycling back to the space 973. To help solids flow from the top of the cones 970 to the spaces 973 under the cone, the pressure of liquid circulating in the space 973 may be regulated to be lower than the pressure above the cone 970. This pressure differential forces the solids to flow through the cone 970 when pressure is applied. To help regulate the pressure in the space 973, a gas space may be present under the cones 970, which provides some compressibility.

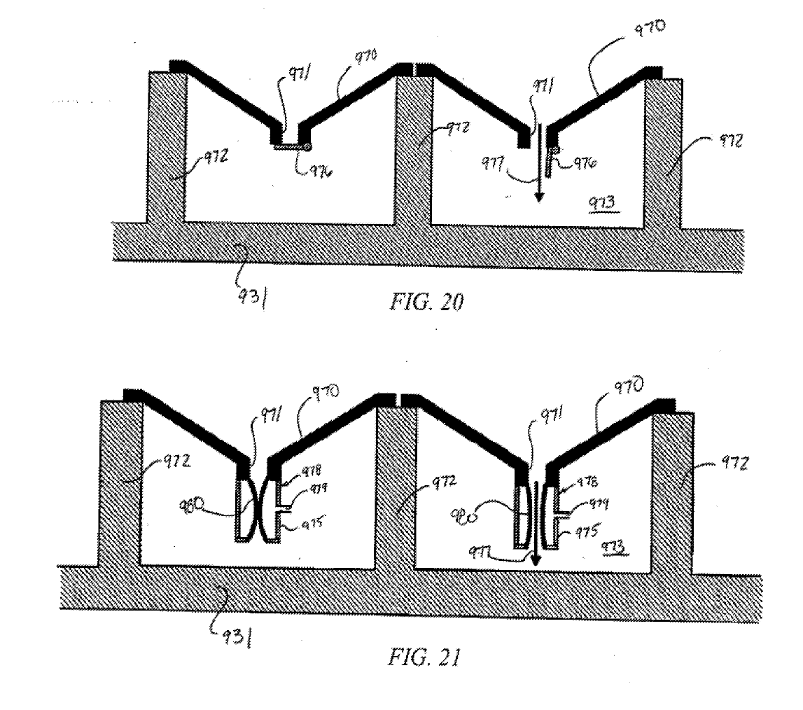

FIG. 20 shows a cone 970, according to an embodiment of the invention. The cone 970 of FIG. 20 has a check valve 976 that allows solids to flow in only one direction through the passage 971. In particular embodiments, the check valve 976 may allows solids to be flushed to the space 973 upon application of pressure, for example, a high-pressure liquid or the like. In other embodiments, the check valve 976 may be actuated using a suitable actuator. Upon opening of the check valve 976, solids may flow through the passage 971 in the direction of arrow 977.

FIG. 21 shows a cone 970, according to an embodiment of the invention. The cone 970 of FIG. 21 has a pinch valve 978 adjacent a passage 971. The center 980 of the pinch valve 978 is flexible rubber and the exterior is a rigid housing 975. When pressure is applied to the space between the rubber center 980 and the rigid housing 975, for example through opening 979, the rubber center 980 closes. When pressure is removed from the space between the rubber center 980 and the rigid housing 975, the pinch valve 978 opens, allowing the flow through the passage 971 in the direction of arrow 977.

FIGS. 22 and 23 illustrates the use of a jet 981 in conjunction with a cone 970, according to embodiments of the invention. In the embodiments of FIGS. 22 and 23, a jet 981 is added to the space above the cone 970. High pressure fluid (e.g., water) exiting the jet 981 forces solids to flow through the passage 971 of the cone 970, allowing a flow indicated by arrow 977 and preventing blockage. As shown in FIG. 22, in particular embodiments the jet 981 may be used in conjunction with a check valve 976. As shown in FIG. 23, in particular embodiments the jet 981 may be used without a check valve 976. In FIG. 23, the pressure above and below the cone 970 (e.g., on each side of the passage 971) may be substantially the same. Accordingly, high-speed fluid (e.g., water) exiting the nozzle 981 forces solids to flow through the passage 971 to the space 973 below the cone 970, where they can be flushed out in a dilute slurry.

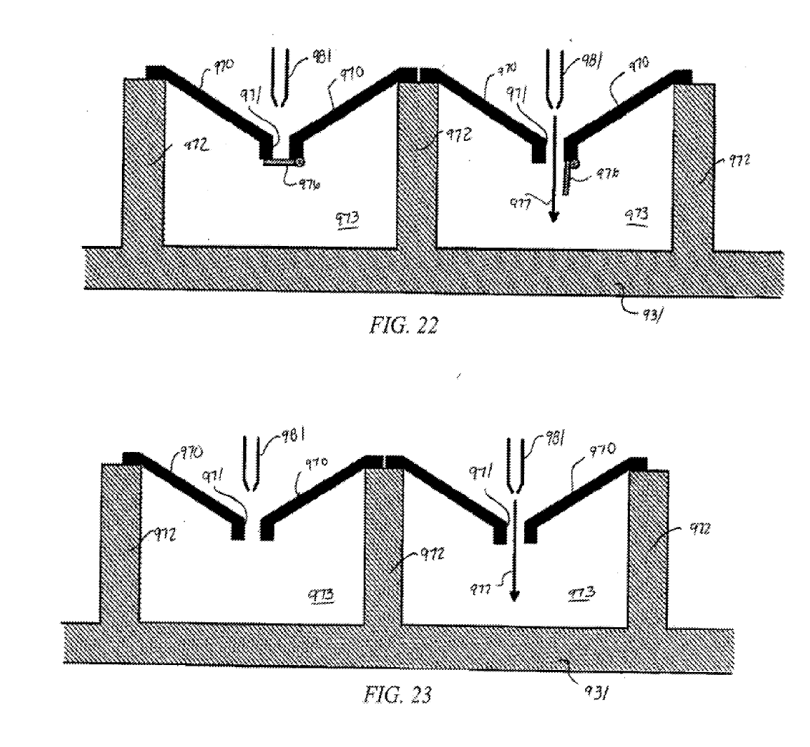

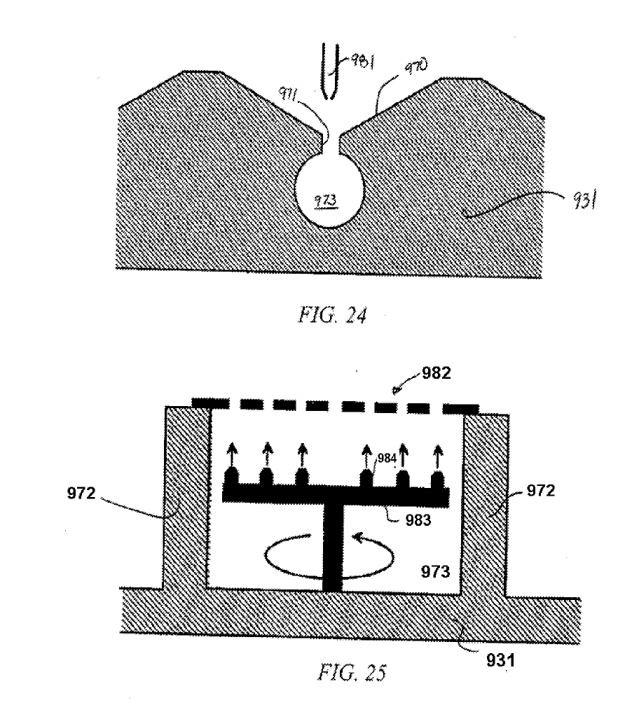

FIG. 24 shows a cone 970 formed in a floor 931, according to an embodiment of the invention. The cone 970 is formed into the floor 931, which as described above may be made of a variety of materials, including, but not limited to, concrete. In such a embodiment, a space 973 may shaped as a pipe in the floor 931 below the tip of the cone 970 to allow removal solids in a dilute slurry. To enhance flow to the space 973, particular embodiments may use a jet 981 to force solids through a passage 971 at the tip of the cone 970.

FIG. 25 shows a grate 982, according to embodiment of the invention. The grate 982 in particular embodiments may be placed in similar location to the cones of other embodiments and operate in similar manner. A rotating set 983 of nozzles 984, which may spray in a manner similar to a lawn sprinkler, blasts solids from the grate 982 allowing the solids to drop to the space 973 below where they may be mixed into a dilute circulating slurry, which brings the solids to the surface for ultimate disposal.

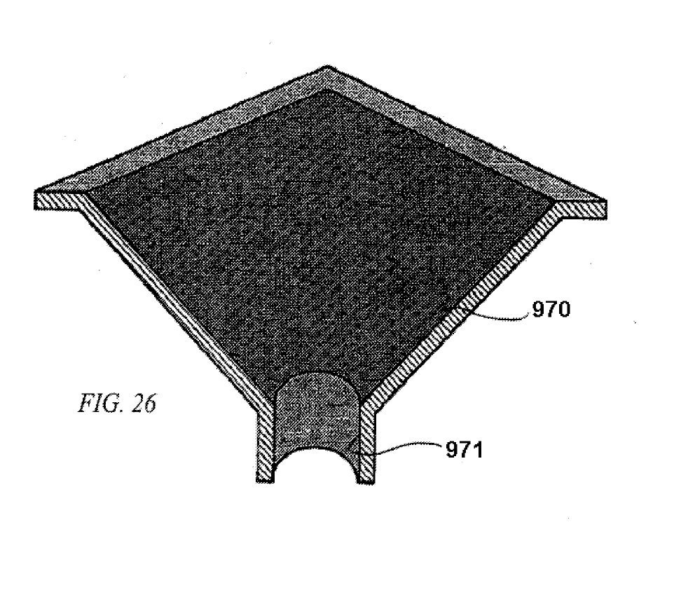

FIG. 26 shows an isometric view of a cut-away cone 970, according to embodiment of the invention. At the tip of the cone is a passage 971.

FIGS. 27, 28, and 29 show patterns for cones, according to embodiments of the invention. FIG. 27 shows a pattern 1001 by which cones 1070 may be laid on the bottom of a chamber that has a rectangular base, according to an embodiment of the invention.

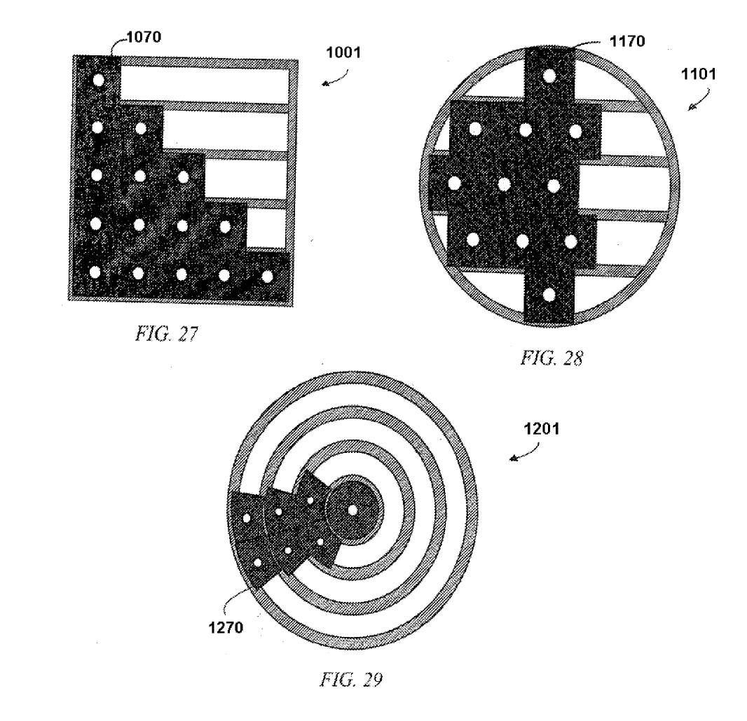

FIG. 28 shows a pattern 1101 by which cones 1170 may be laid on the bottom of a chamber that has a circular base, according to an embodiment of the invention.

FIG. 29 shows a pattern 1201 by which cones 1270 may be laid on the bottom of a chamber that has a circular base, according to an embodiment of the invention. The pattern 1201 is shown as a series of concentric cones 1270. In other embodiments, the pattern may be a spiral of cones.

Although the present invention has been described with several embodiments, a myriad of changes, variations, alterations, transformations, and modifications may be suggested to one skilled in the art, and it is intended that the present invention encompass such changes, variations, alterations, transformation, and modifications as they fall within the scope of the appended claims.

What is claimed is:

1. A method for processing biomass comprising:
forming a first pile comprising biomass;
inoculating said first pile comprising biomass;
circulating a liquid in said first pile;
fermenting the biomass in said first pile to produce a carboxylate salt; and
extracting at least a portion of the liquid in the first pile and passing said portion of liquid to a second pile comprising biomass, wherein said second pile is operating at a higher carboxylate salt concentration than said first pile.

2. The method of claim 1 further comprising introducing calcium carbonate into the first pile while said first pile comprising biomass is being formed.

3. The method of claim 1 wherein circulating a liquid in said first pile is performed intermittently.

4. The method of claim 1 further comprising pretreating the biomass prior to inoculating said first pile comprising biomass, wherein pretreating and fermenting said biomass take place in the same enclosure without transferring the biomass.

5. The method of claim 4 wherein pretreating the biomass comprises introducing into the biomass lime or lime and air.

6. The method of claim 5 wherein said air is scrubbed of carbon dioxide prior to being introduced into the biomass.

7. The method of claim 1 further comprising handling a multiplicity of piles comprising biomass in a round-robin manner.

8. The method of claim 1 further comprising regulating the temperature of the circulating liquid in said first pile.

9. The method of claim 8 wherein regulating the temperature of the circulating liquid in said first pile comprises passing said circulating liquid through a heat exchanger.

10. The method of claim 1 further comprising controlling the temperature of the biomass in said first pile by regulating the temperature of the circulating liquid in said first pile.

11. A method of processing biomass, the method comprising:
transporting biomass to a chamber to form a biomass pile, the chamber defined by at least a bottom and adjustable cover;
transferring a liquid to the chamber;
lowering the adjustable cover from a raised position to a lowered position;
fermenting the biomass pile in the chamber to produce a carboxylate salt while the adjustable cover is in the lowered position; and
extracting at least a portion of the liquid from the biomass pile and passing the portion of liquid to a second pile comprising biomass, wherein said second pile is operating at a higher carboxylate salt concentration than the biomass pile.

12. The method of claim 11, wherein processing the biomass includes fermenting the biomass in the presence of an inoculum.

13. The method of claim 11, wherein the adjustable cover in the lowered position is adjacent the biomass pile.

14. The method of claim 11, further comprising:
supporting the adjustable cover with a pole that has perforations, wherein transporting the biomass to the chamber to form the biomass pile and transferring fluids to the chamber are carried out through the perforations.

15. The method of claim 11, wherein lowering the adjustable cover from a raised position to a lowered position is carried out by a winch located on the pole, the winch attached to the adjustable cover via a cable and releasing the cable to lower the adjustable cover.

16. A method for processing biomass comprising:
forming a first pile comprising biomass;
inoculating the first pile;
contacting a fluid with the first pile;
fermenting the biomass in the first pile to produce a carboxylate salt; and
collecting at least a portion of the fluid from the first pile, the fluid comprising at least some of the carboxylate salt, and passing the portion of fluid to a second pile comprising biomass.

17. The method of claim 16 further comprising introducing calcium carbonate into the first pile while said first pile comprising biomass is being formed.

18. The method of claim 16 wherein contacting the fluid with the first pile is performed intermittently.

19. The method of claim 16 further comprising pretreating the biomass prior to inoculating said first pile comprising biomass, wherein pretreating and fermenting said biomass take place in the same enclosure without transferring the biomass.

20. The method of claim 19 wherein pretreating the biomass comprises introducing into the biomass lime or lime and air.
